(19) United States
(12) Patent Application Publication
(10) Pub. No.: US 2003/0162756 A1
(43) Pub. Date: Aug. 28, 2003

(54) Use of 2-hydroxy or 2-acetyloxy-4-trifluoromethylbenzoic acid derivatives as an agent for the treatment and prevention of mild cognitive impairment

(75) Inventors: Julian Garcia-Rafanell, Barcelona (ES); Genis Munoz Orti, Barcelona (ES); Jose Carlos Navas Ramirez, Barcelona (ES); Jose Ignacio Izquierdo Pulido, Barcelona (ES)

Correspondence Address: SCULLY, SCOTT, MURPHY & PRESSER, 400 Garden City Plaza, Garden City, NY, 11530, US

(73) Assignee: J. Uriach & CIA. S.A., Plegamans (ES)

(21) Appl. No.: 10/313,498

(22) Filed: Dec. 6, 2002

(30) Foreign Application Priority Data
Dec. 7, 2001 (ES) 200102725

(51) Int. Cl. A61K 31/60

(52) U.S. Cl. 514/159

(57) ABSTRACT

The present invention relates to the use of 2-hydroxy or 2-acetyloxy-4-trifluoromethylbenzoic acid, respectively, for the treatment or prevention of mild cognitive impairment (MCI) in mammals, especially in human beings. Futhermore, the present invention also concerns the use of 2-hydroxy or 2-acetyloxy-4-trifluoromethylbenzoic acid, respectively, for delaying or preventing the conversion of MCI into dementia, especially Alzheimer's Disease.

### FIELD OF THE INVENTION

The present invention relates to the use of 2-hydroxy and 2-acetyloxy-4-trifluoromethylbenzoic acid derivatives, respectively, for the treatment and prevention of mild cognitive impairment in mammals, especially in human beings. Futhermore, the present invention also concerns the use of 2-hydroxy and 2-acetyloxy-4-trifluoromethylbenzoic acid derivatives, respectively, for delaying or preventing the conversion of mild cognitive impairment into dementia, especially Alzheimer's Disease.

### DESCRIPTION OF THE PRIOR ART

Mild cognitive impairment (MCI) is a term used to describe memory decline or other specific impairment in individuals who do not have dementia or significant impairment of other cognitive functions beyond that expected for their age and education. It has been suggested that as many as 38% of the elderly population would meet criteria for MCI and although the associated memory deficits are mild, the fact that up to 15% of MCI patients develop Alzheimer's disease (AD) annually has prompted serious attention.

Despite the above-mentioned high conversion rate into AD, MCI is different from early or mild AD, as patients with AD are impaired not only in memory perfomance but in other cognitive domains as well; and moreover they meet diagnostic criteria for actual dementia. Diagnostic criteria for MCI are based on cut-off scores on memory tests; however, until recently physicians and researchers had difficulty in diagnosing MCI.

Experts agree that five characteristic criteria for MCI can be suggested:

Memory complaints

Abnormal memory for age

Ability to carry out normal activities of daily living

Normal general cognitive functions

Absence of dementia

The U.S. Food and Drug Administration (FDA) has recently stated clearly that mild cognitive impairment “is a condition separate from Alzheimer's disease”. The FDA further underlined that MCI constitutes a valid target for new drug therapies, regardless of whether a particular drug also slows the progression to dementia. Experts agree that it would prove necessary to provide specific biological markers to distinguish the separate situation of each of the two disorders.

At present there are no medicaments approved for the specific treatment of MCI. Currently, clinical trials are in progress in MCI patients with acetylcholinesterase inhibitors. Other strategies being considered by experts for the treatment of MCI comprise free radical scanvengers and anti-oxidants, oestrogen replacement therapy, as well as a few novel strategies, such as anti-amyloid agents and nerve growth factor.

2-Acetyloxy-4-trifluoromethylbenzoic acid, better known by its international non-proprietary name (INN) Triflusal, is a platelet aggregation inhibitor marketed for the treatment of thrombo-embolic diseases under the Trademark Disgren®. Its main metabolite, 2-hydroxy-4-trifluoromethylbenzoic acid (also known by the acronym HTB), also possesses a remarkable activity as platelet anti-aggregant. Both compounds are described in U.S. Pat. No. 4,096,252.

### DESCRIPTION OF THE INVENTION

The present invention is based on the surprising finding that triflusal as well as its metabolite, HTB, are also effective in the prevention and/or treatment of MCI. In particular, triflusal and HTB are effective to alleviate MCI, to delay the progression of MCI and/or to stabilize MCI (i.e. to prevent further deterioration in the condition of MCI patients). Futhermore, the present invention is based on the surprising finding that triflusal as well as HTB are useful for delaying the conversion of MCI into dementia, especially Alzheimer's disease, and/or for preventing the conversion of MCI into dementia, especially Alzheimer's disease.

Triflusal and HTB can be depicted generically by the following Formula (I)

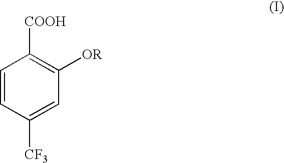

wherein R is hydrogen or COCH₃.

According to a preferred embodiment of the present invention R is COCH₃, wherein this embodiment is designated Triflusal. According to a further preferred embodiment R is H; here the resulting compound is designated HTB.

It was an object according to the present invention to provide compounds useful for the production of medicaments for treating or alleviating MCI or preventing MCI as well as for delaying or preventing the conversion of MCI into dementia, especially Alzheimer's disease.

This object is solved by use of the compounds according to Formula (I) as described above, or a pharmaceutically acceptable salt or a prodrug thereof, for the preparation of a medicament for the treatment, including alleviation, or prevention of MCI, as well as for the delay or prevention of the conversion of MCI into dementia, especially Alzheimer's disease. According to a preferred embodiment, a therapeutically effective amount of a compound of Formula (I) or a pharmaceutically acceptable salt or a prodrug thereof is incorporated in a pharmaceutical composition, wherein said composition is capable of treatment, alleviation or prevention of MCI. Furthermore it is possible to provide a pharmaceutical composition comprising a compound of Formula (I) or a pharmaceutically acceptable salt or a prodrug thereof for the delay or prevention of the conversion of MCI into dementia, especially Alzheimer's disease.

The pharmaceutically acceptable salts of a compound of Formula (I) include any of the salts commonly used in pharmaceutical chemistry, such as for example the salts with inorganic cations such as sodium, potassium, calcium, magnesium, lithium, aluminium, zinc, etc., as well as the salts formed with ammonia and other pharmaceutically acceptable amines.

Throughout the present description, the term “prodrug” of a compound of Formula (I) means any precursor compound of Formula (I) that is capable of being metabolised and releasing in vivo a compound of Formula (I), that is Triflusal or HTB. Such a compound is, for example, disclosed in ES 2 021 258.

The processes for preparing Triflusal or HTB are disclosed in the above-mentioned U.S. Pat. No. 4,096,252.

The compounds of Formula (I) can be used to treat or alleviate MCI in mammals, preferably human beings, or to prevent the occurrence of MCI. Furthermore, the compounds of Formula (I) can be used to delay or prevent the conversion of MCI into dementia, especially Alzheimer's disease.

The compounds according to Formula (I) delay or decrease the progression of mild cognitive impairment (i.e., delay or decrease the further impairment or deterioration of cognitive function in MCI patients). For example, in the whole population or in a subset of the population, a reduction in the cognitive function between the beginning and the end of the study that is smaller in the group of patients receiving triflusal than in the group of patients receiving placebo is observed. This result can be defined as delay in the progression of mild cognitive impairment.

Moreover, the compounds according to Formula (I) stop the progression of mild cognitive impairment. For example, in the whole population or in a subset of the population receiving triflusal, the same cognitive function at the beginning and at the end of the study can be observed, while in the group of patients receiving placebo, a decrease in the cognitive function is observed. This result can be defined as stabilization of mild cognitive impairment.

The compounds of Formula (I) can also be used to delay the onset of dementia, especially Alzheimer's disease, in the subgroup of patients with MCI that develop dementia within a certain period of time. Here a binary variable, “conversion into dementia”, is used. It is observed that in the group of patients receiving triflusal, the time elapsed from the inclusion of the patient in the study until the diagnosis of dementia is longer than the time elapsed for such dementia diagnosis in the placebo group. This result can be defined as delay of the conversion of MCI into dementia, especially Alzheimer's disease.

Futhermore, the compounds of Formula (I) can also be used to avoid or prevent that a percentage of patients with MCI progress into dementia, especially Alzheimer's disease, within a certain period of time. Here the variable “conversion into dementia” is the main variable. The effect observed is that in the group of MCI patients receiving triflusal, the percentage of subjects that evolve into demented patients (i.e. develop dementia) is lower than in the placebo group.

The dose of the compound of Formula (I) necessary to treat or alleviate or delay or stabilize or prevent MCI or to delay or prevent conversion of MCI into dementia, especially Alzheimer's disease, will depend on a variety of factors such as the severity of the symptoms, the age and the body weight of the patient as well as the chosen route of administration. Any person skilled in the art will be in a position to readily determine the appropriate dosage, depending on these factors, without having to incur any undue experimentation. In human therapy, dosage will generally be in the range of between about 100 mg and about 3000 mg daily of a compound of Formula (I), which can be administered in one or several dosage units. Depending on the particular condition to be treated and the patient situation, however, doses outside this range might be needed, which, as mentioned above, may be readily determined by those skilled in the art, without requiring undue experimentation.

The compounds of Formula (I) can also be given in combination with one or more other active ingredients such as neurotransmitter modulators (e.g. acetylcholinesterase inhibitors, NMDA receptor antagonists,etc), antioxidants or free radical scavengers, nootropic agents, inhibitors of beta-amyloid protein production or deposition, antiinflammatory agents, neurotropic agents and modulators of secondary risk factors (e.g. antihypertensive agents, statins, hormone replacement therapy, etc). The compounds may be formulated in a single dosage unit or may be provided as separate formulated products to be used in simultaneous, sequential or separated treatment.

The compounds of Formula (I) can be administered in the form of any pharmaceutical formulation, the nature of which shall depend, as is well known, on the route of administration and the actual use for which it is formulated. These pharmaceutical compositions can be prepared by conventional methods, using compatible pharmaceutically acceptable excipients or vehicles. Examples of such compositions include capsules, tablets, syrups, powders and granulates for the preparation of extemporaneous solutions, injectable preparations, etc. A preferred route of administration for the compounds of Formula (I) is the oral route. For example, they can be administered as hard gelatine capsules containing e.g. 50, 100, 200, 300, 400 or 500 mg of the compound of Formula (I) or a pharmaceutically acceptable salt or prodrug thereof.

### EXAMPLES

The invention may be illustrated by carrying out clinical investigations using conventional methodologies well known to those skilled in the art. Typically, for such clinical trials the following can be mentioned:

i) Design of study: multicentric, randomised, double-blind, with parallel groups and placebo-controlled study.

ii) Method:

total number of patients to be recruited: minimum of about 500.

subjects included in the study: patients from both sexes, aged between 65 and 90 years and diagnosed with MCI;

criteria to diagnose MCI: patients will be diagnosed with MCI and will be thus eligible for the study if they fulfil the following requirements: a) memory impairment complaint of the patient; b) objective memory impairment, as determined using known, reliable and well-established memory tests; c) normal general cognitive function, as assessed using known, reliable and well-established global cognitive function tests; d) intact activities of daily living, as assessed using known, well-established tests for this purpose, and e) absence of dementia, assessed using extensively accepted criteria to diagnose dementia, such as the current criteria of Diagnostic and Statistical Manual of Mental Disorders (DSM) or of the National Institute of Neurological and Communicative Disorders and Stroke-Alzheimer's Disease and Related Disorders of Association (NINCDS-ADRDA).

end-points of the study: a) to evaluate the efficacy of triflusal versus placebo to stabilize or delay the progression of MCI; this will be done by determining cognitive impairment using an objective assessment, i.e. known, well-established tests to evaluate cognitive function; and b) to evaluate the efficacy of trifusal to delay the conversion of MCI into dementia and especially Alzheimer's disease, assessed by means of known, extensively accepted neuropsychological and functional tests.

drug treatment: patients will be randomised to receive triflusal at a dose within the range of 300 to 1200 mg/day or placebo for up to 48 months.

As an example for a protocol for such a study, the following can be provided:

“Clinical trial to evaluate the efficacy of triflusal versus placebo in patients with mild cognitive impairment (MCI)”

### TYPE OF STUDY AND DESIGN THEREOF

1. Aims of the study

Main aim: to evaluate the efficacy of triflusal versus placebo to stabilize or delay the progression of mild cognitive impairment (MCI)

Secondary aims: to evaluate the efficacy of triflusal versus placebo to delay conversion of MCI into dementia, especially Alzheimer's disease; to evaluate the efficacy of triflusal versus placebo to delay functional impairment.

2. Study Phase

Phase IV study. Minimum number of patients: about 500.

3. Type of Control

Parallel-, double blind-study where efficacy and safety of triflusal and/or HTB will be compared to placebo.

Each group of patients will receive one of the following two treatments under random assignment:

a) Triflusal 900 mg: 1 capsule 300 mg in the morning and 2 capsules at lunch time

b) Placebo: 1 capsule in the morning and 2 capsules at lunch time

4. Patient Selection

4.1 PATHOLOGY UNDER STUDY

Mild cognitive impairment. Diagnosis will be established according to the criteria of the American Academy of Neurology (REF 1)

4.2 INCLUSION AND EXCLUSION CRITERIA

Inclusion criteria:

1. Patients from both sexes aged 65-90, both included

2. Patient must have attended school for at least 4 years

3. Availability of an informant to attend all scheduled visits together with the patient

4. Diagnosed with MCI:

Memory impairment complaint of the patient, preferably corroborated by a reliable informant

Objective memory impairment, determined by a score in the deferred verbal memory test of the Barcelona test (REF 2) that must be at least 1.5 SD (standard deviation) lower than in the corresponding normal population. Alternative memory impairment tests known in the art that can also be used comprise the Buschke Free and Cued Selective Reminding Test; the Logical Memory subtest of the Wechsler Memory Scale (WMS); and the Digit Symbol Substitution test.

Normal global cognitive function, measured objectively by a score in the adjusted MMSE test≧24.

Intact activities of daily living, measured objectively by a score in the Blessed Dementia Rating Scale, part A<4

Absence of dementia according to the DSM-IV criteria

Exclusion criteria:

1. Serious psychiatric illness: in case patient is receiving treatment against depression and/or anxiety, patient must have remained stable for the last three months. In a patient not receiving antidepressant therapy, a score≧11 in the Hamilton Depression Rating Scale (REF 3) means that the patient would require antidepressant therapy and hence the patient cannot be included in the study until at least three months thereafter.

2. Mental deficiency, illiteracy, unability to understand and/or perform the study.

3. Neurological pathology that may cause to cognitive symptoms

4. Evidence of significant vascular pathology, as evidenced by a score in the Hachinski's ischemia scale (REF 4) >4

5. Presence of images in the brain Magnetic Resonance Imaging that may point towards vascular dementia. Specifically:

a) Large vessel infarctions. This includes anterior, middle and posterior cerebral artery infarction, as well as vertebro-basilar territory infarctions.

b) Border zone infarctions

c) Small vessel vascular impairment, as shown by at least 2 lesions in the basal ganglia, and at least 2 lacunar lesions (>2 mm) in the frontal white matter.

d) Small vessel vascular impairment characterized by white matter lesions (hyperintensities) in the frontal and parietal regions that are confluent (level 3 of the “Age-Related White Matter Changes”[ARWMC]) in at least two of said regions and which begin to be confluent (level 2 ARWMC) in the remaining areas

e) Bilateral thalamic lesions (at least two lesions in each thalamus, and beyond 1 cm from third ventricle)

6. Systemic disease, whether serious and/or associated to cognitive impairment

7. Contraindication to NSAID treatment

8. Chronic treatment with acetylsalicylic acid or other antiplatelet agents, triflusal, NSAIDs, oral anticoagulants, acetylcholinesterase inhibitors

9. Alcoholism or drug addiction

10. Having participated in a clinical trial within the three previous months

11. Serious allergic disorder (e.g. asthma)

12. Life expectancy lower than the duration of the study

13. Patients with a history of brain haemorrhage, peptic ulcer or hypersensitivity to salicylates.

4.3 NUMBER OF SUBJECTS AND JUSTIFICATION THEREOF

The main evaluation variable is the difference between groups regarding the change in the score in the ADAS-cog test.

In studies carried out in patients with established Alzheimer's disease where the efficacy of various drugs has been evaluated, differences of about 2.5 points in the ADAS-cog test after 6 months of treatment have been observed between the placebo- and the drug-treated groups (REF 5). The slope of cognitive impairment versus time should be less pronounced in patients with MCI than in patients with Alzheimer's disease, for which reason in the present study a follow up period of 18 months has been selected.

We regard a decrease in the ADAS-cog score of 2.5 points compared to the placebo group as clinically significant and we assume a standard deviation of 10, according to the values observed in a similar study. Assuming a first class risk of a bilateral 5% and a statistical power of 80%, 253 patients are needed in each treatment group in order to detect differences equal or higher than those previously mentioned. Considering a loss/withdrawal rate of 10% (253=x−(x/10)), the number of patients to be recruited is of 282 in each group, 564 in total.

4.4 WITHDRAWAL CRITERIA AND ANALYSIS OF WITHDRAWALS/EXCLUSIONS

Patient can freely withdraw from the study at any time.

Onset of other diseases that may have a significant effect on the evaluation of the clinical situation of the patient, according to the investigator in charge.

Clinically relevant adverse events.

Patients who do not collaborate with the performance of the study.

Diagnosis of dementia according to the DSM-IV criteria.

Onset of a disease which requires drug treatment that is incompatible with the protocol.

Unsatisfactory treatment compliance (<85%, >115%), including use of medication or substances forbidden in the protocol.

5. Description of Drug Treatment

5.1 TREATMENT AND DOSE

Period of treatment for 18 months:

| | |
|---|---|
| Experimental drug: | TRIFLUSAL or HTB |
| Composition: | Each capsule contains 300 mg |
| | triflusal or equivalent |
| Dosage: | One capsule (300 mg) in the |
| | morning + 2 capsules (600 mg) at |
| | lunch time |
| Pharmaceutical form: | Hard gelatine capsules |
| Route of administration: | Oral |
| Control: | PLACEBO |
| Composition: | Each capsule contains 300 mg |
| | mannitol |
| Dosage: | One capsule in the morning + 2 |
| | capsules at lunch time |
| Pharmaceutical form: | Hard gelatine capsules |
| Route of administration: | Oral |

Since it is blind study, capsules will have identical organoleptic properties.

5.2 CRITERIA TO MODIFY TREATMENT DURING STUDY

Patients will take medication each day throughout the study. In case of gastric intolerance, patient will be allowed to divide the lunch dosis (one capsule at lunchtime and one capsule at dinner time). If a patient interrupts drug treatment for more than 15 days, he will be withdrawn from the study.

5.3 CONCOMITANT TREATMENTS

a) Allowed treatments:

All concomitant medication taken by the patient,whether prescribed by a doctor or not, will be indicated in the Case Report Form (CRF). In case the patient requires an analgesic agent, he can take paracetamol or metamizole. Should treatment with NSAIDs be required, it will be necessary to note in the CRF the therapeutical indication, drug, dose and treatment duration.

b) Excluded treatments:

The concomitant use of the following drugs must be avoided:

Anticoagulant agents: acenocoumarol, warfarin.

Acetylsalicylic acid or other antiplatelet agents/NSAIDs.

Due to the long duration of the study and the profile of the subjects to be included, the use of antiplatelet agents/NSAIDs may be allowed exceptionally. In any case, duration of treatment cannot be over 1 month, or in case it is a discontinued treatment, the global duration of the treatment cannot exceed 3 months.

In case patient requires treatment for a longer period than the one established as allowable, he will be withdraw from the study but followed up until the end thereof. These patients will be included in the intention-to-treat analysis.

6. Course of the Study and Evaluation of the Response

6.1 EVALUATION OF THE RESPONSE

6.1.1 MAIN EVALUATION VARIABLE

Cognitive impairment objectively assessed by the score in the ADAS-cog test

6.1.2 SECONDARY EVALUATION VARIABLE

The criteria of conversion to dementia will follow the criteria according to DSM-IV plus psychometric criteria based on the following tests:

ADAS-cog (REF 6)

Mini-Mental State Examination (REF 7)

Blessed Dementia Rating Scale (part A) (REF 8)

Clinical Dementia Rating Scale (REF 9,10)

CLOX test (REF 11)

Buschke cued recall selective reminding test (REF 12)

Rey Complex Figure (REF 13,14,15)

Digit Symbol Substitution test (REF 16)

Verbal fluency test (REF 17)

Benton temporal orientation test (REF 18)

Trail Making test (REF 19)

PET (Positron emission tomography)

This will be performed at baseline (visit 1) and at the end of the study (visit at month 18, or when the patient is diagnosed with dementia). This will be performed using a high resolution equipment (FWHM=4mm), ECAT EXACT HR+ (Siemens, CTI). As radiotracer ¹⁸Fluoro-labeled fluoro-desoxy-glucose (¹⁸F-FDG) will be used. Regions of interest on the PET image will be delimited, including among others, hippocampus, and lateral temporal, parietal, posterior cingulate and frontal cortex in both hemispheres. Glucose metabolic consumption for each region of interest will be calculated using the Sokoloff mathematical model (ml/100 gr/min). Moreover, images will be analysed using the Statistical Parametric Mapping (SPM99; Friston) software comparing both groups (triflusal vs. placebo). This technique will only be applied to a subgroup of 50 patients in the study, in previously agreed centres.

Brain Magnetic Resonance Imaging (MRI)-volumetry and MRI-spectroscopy This will be performed at baseline (visit 1) and at the end of the study (visit at month 18, or when the patient is diagnosed with dementia). Volumetric and spectroscopic studies using brain MRI-spectroscopy will be carried out measuring volume and metabolites in several regions of interest as potential markers of the risk of progression of MCI into dementia, and to evaluate possible differences between groups (triflusal vs. placebo). As regions of interest, the following will be included, among others: entorhinal cortex, hippocampus, cingulum and cortex delimiting the superior temporal sulcus. This technique will only be applied to a subgroup of 150 patients in the study, in previously agreed centres.

Genetic risk markers: polymorphism in the apolipoprotein E, alpha 2 macroglobulin and interleukin-1A genes. Determination will be carried out under blind conditions in a centralized centre.

Impact of the pathology under study upon quality of life of the patient and the patient's care giver. Patient and care giver will be asked to answer a questionnaire directed to the impact of patient's MCI status upon their quality of life.

Safety of the treatment: Evaluation of safety will be based upon the follow up of adverse events as well as control analysis. Control analysis will be carried out at the beginning of the study and at months 6 and 18 (final), and it will include hematology, biochemistry and electrolites.

6.2 COURSE OF THE STUDY

6.2 1 Control visits

Visits will be scheduled taking the randomization visit as reference for calculation purposes.

Visit 1 (between week-2 and week 0). Screening

The following are to be studied:

Signature of written consent

Demographic profile of the patient (sex, race, birth date . . . ).

Complete clinical history including clinically relevant events during the last two years.

Evaluation of selection criteria that may already be answered.

Performance of neuroimaging studies: simple MRI; MRI-spectroscopy and PET in the subgroup of patients.

Blood sample for analysis (analysis carried out during the preceding 30 days will be accepted).

Study of biological markers (optional)

Visit 2 (basal, week 0). Randomization

he following are to be checked:

Physical examination.

Concomitant medication from one month before inclusion in the study.

Completion of the neuropsychological test battery (started during the Screening visit)

Relevant results from the laboratory analysis will be indicated.

All selection criteria must be fulfilled.

Patient will be informed to start taking the medication that day.

Relevant data from neuroimaging studies will be indicated.

Visit 3 (month 1±7 days)

The following is to be checked:

Changes in concomitant medication since last visit.

Patient will be asked about adverse events

If the investigator considers that the patient has converted into dementia, the corresponding section in the CRF will be completed (with information about tests supporting this decision) and the patient will be withdrawn from the study.

Visit 4 (month 3±7 days)

The following is to be checked:

Changes in concomitant medication since last visit.

Patient will be asked about adverse events

If the investigator considers that the patient has converted into dementia, the corresponding section in the CRF will be completed (with information about tests supporting this decision) and the patient will be withdrawn from the study.

Visit 5 (month 6±7 days)

The following is to be checked/carried out:

Changes in concomitant medication since last visit.

Patient will be asked about adverse events

Obtain a blood sample for analysis in a local laboratory

Neuropsychological test battery

If the investigator considers that the patient has converted into dementia, the corresponding section in the CRF will be completed (with information about tests supporting this decision) and the patient will be withdrawn from the study.

Visit 6 (month 9±7 days)

The following is to be checked:

Changes in concomitant medication since last visit.

Patient will be asked about adverse events

If the investigator considers that the patient has converted into dementia, the corresponding section in the CRF will be completed (with information about tests supporting this decision) and the patient will be withdrawn from the study.

Visit 7 (month 12±7 days)

The following is to be checked/carried out:

Changes in concomitant medication since last visit.

Patient will be asked about adverse events

Neuropsychological test battery

If the investigator considers that the patient has converted into dementia, the corresponding section in the CRF will be completed (with information about tests supporting this decision) and the patient will be withdrawn from the study.

Visit 8 (month 15±7 days)

The following is to be checked:

Changes in concomitant medication since last visit.

Patient will be asked about adverse events

If the investigator considers that the patient has converted into dementia, the corresponding section in the CRF will be completed (with information about tests supporting this decision) and the patient will be withdrawn from the study.

Visit 9 (month 18±7 days). End of study

The following is to be checked/carried out:

Changes in concomitant medication since last visit.

Patient will be asked about adverse events

Obtain a blood sample for analysis in a local laboratory

Neuropsychological test battery

Neuroimaging study in the subgroup of patients (MRI-spectroscopy and PET)

The section corresponding to the completion of the study shall be filled in.

The above information is only intended for illustration purposes and shall not be construed as limiting the scope of the present invention in any way.

### REFERENCES

1. Petersen R C, Stevens J C, Ganguli M, Tangalos E G, Cummings J L, DeKosky S T. Practice parameter: early detection of dementia: mild cognitive impairment (an evidence-based review). Report of the Quality Standards Subcommittee of the American Academy of Neurology. Neurology 2001; 56:1133-1142

2. J. Pena-Casanova. Programa integrado de exploración neuropsicológica-test Barcelona: bases teóricas, objetivos y contenidos. Rev. Logop., Fon., Audiol., vol Xl, n^{o} 2(66-79),1991

3. Hamilton M. Development of a rating scale for primary depressive illness. British Journal of Social and Clinical Psychology. 1967; 6:278-296

4. Hachinski V C et al. Cerebral blood flow in dementia. Arch Neurol 1975; 32: 632-637

5. Rösler M, Anand R, Cicin-Sain A, Gauthier S, Agid Y, Dal-Bianco P et al. Efficacy and safety of rivastigmine in patients with Alzheimer's disease:

international randomised controlled trial. BMJ, 1999, 318: 633-640

6. Rosen W G, Mohs R C, Davis K L. A new rating scale for Alzheimer's disease. Am J Psychiatry. 1984 141(11):1356-1364

7. Folstein M F, Folstein S E, McHugh P R. Mini-mental state. A practical method for grading the cognitive state of patients for the clinician. J Psychiatr Res 1975; 12:189-198

8. G. Blessed et al. The association between quantitative measures of dementia and of senile change in the cerebral grey matter of elderly subjects. Br J Psychiatry, 1968, 114: 797-811

9. Hughes C P, et al. A new clinical scale for the staging of dementia. Br J Psychiatr 1988;140:566-572

10. Morris J. The CDR: current version and scoring rules. Neurology 1993;43:2412-2413

11. Royall et al. CLOX: an executive clock drawing task. J Neurol Neurosurg Psychiatry 1998;64:588-594

12. Buschke H. Cued recall in amnesia. Journal of Clinical Neuropsychology. 1984; 6 (4): 433-440

13. L'examen psychologique dans les cas d'encephalopathie traumatique. Archives de Pscyhologie. 1941;28:286-290

14. Osterrieth P A, et al. Le test de copie d'une figure complexe. Archives de Psychologie. 1944;30:206-356

15. J. Corwin and F. W. Bylsma. The Clinical Neuropsychologist. 1993;7:9-15

16. Weschler D. 1981. Manual for the Weschler Adult Intelligence Scale. Psychological Corporation, New York. Weschler Adult Intelligence Scale (WAIS-III) 3rd edition. Administration and Scoring Manual by the Psychological Corporation, San Antonio, Tex., USA

17. Benton A L and Hamsher R de S. Multilingual Aphasia Examination. Iowa City, Iowa: ASA Associates

18. Benton A L. Neuropsychological assessment. Annu Rev Psychol. 1994; 45: 1-23

19. Armitage S G. Psychol Monogr series 1, n^{o} 227; 1-48 (1946-69)

1. Use of a compound of Formula (I) 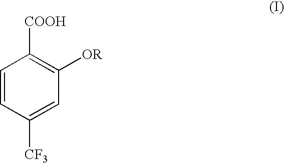
wherein R represents hydrogen or COCH₃ or a pharmaceutically acceptable salt or a prodrug thereof for the manufacture of a medicament useful for the treatment of mild cognitive impairment.

2. Use of a compound of Formula (I) 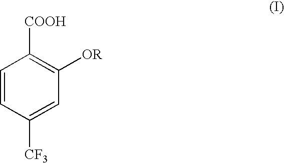
wherein R represents hydrogen or COCH₃ or a pharmaceutically acceptable salt or a prodrug thereof for the manufacture of a medicament useful for the prevention of mild cognitive impairment.

3. Use of a compound of Formula (I) 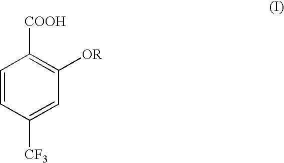
wherein R represents hydrogen or COCH₃ or a pharmaceutically acceptable salt or a prodrug thereof for the manufacture of a medicament useful for the alleviation of mild cognitive impairment.

4. Use of a compound of Formula (I) 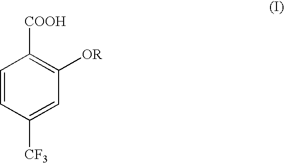
wherein R represents hydrogen or COCH₃ or a pharmaceutically acceptable salt or a prodrug thereof for the manufacture of a medicament useful for the stabilization of mild cognitive impairment.

5. Use of a compound of Formula (I) 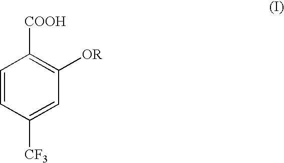
wherein R represents hydrogen or COCH₃ or a pharmaceutically acceptable salt or a prodrug thereof for the manufacture of a medicament useful for delaying the progression of mild cognitive impairment.

6. Use of a compound of Formula (I) 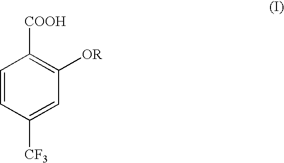
wherein R represents hydrogen or COCH₃ or a pharmaceutically acceptable salt or a prodrug thereof for the manufacture of a medicament useful for delaying conversion of mild cognitive impairment into dementia.

7. Use according to claim 6, wherein dementia is Alzheimer's disease.

8. Use of a compound of Formula (I) 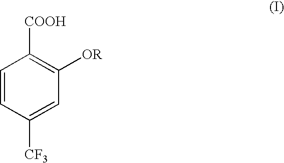
wherein R represents hydrogen or COCH₃ or a pharmaceutically acceptable salt or a prodrug thereof for the manufacture of a medicament useful for the prevention of the conversion of mild cognitive impairment into dementia.

9. Use according to claim 8, wherein dementia is Alzheimer's disease.

10. Use according to any of claims 1 to 9, wherein R represents COCH₃.

11. Use according to any of claims 1 to 9, wherein R represents hydrogen.
